(19) United States
(12) Patent Application Publication
(10) Pub. No.: US 2019/0038390 A1
(43) Pub. Date: Feb. 7, 2019

(54) NON-INVASIVE DEVICE AGAINST URINARY INCONTINENCE

(71) Applicant: MELO S.R.L., Brescia (IT)

(72) Inventors: Giancarlo Donati, Rogno (IT); Ernesto Fardelli, Rogno (IT); Massimo Spagnoli, Castiglione delle Stiviere (IT)

(21) Appl. No.: 16/075,491

(22) PCT Filed: Feb. 3, 2017
(86) PCT No.: PCT/IB2017/050588

§ 371 Date: Aug. 3, 2018

(30) Foreign Application Priority Data
Feb. 5, 2016 (IT) 102016000012205

(51) Int. Cl. A61F 2/00

(52) U.S. Cl. CPC A61F 2/0054; A61F 2250/0018; A61B 17/122; A61F 2230/0013; A61F 2220/0083

(57) ABSTRACT

A non-invasive device against urinary incontinence is described, which is adapted to be applied to the external part of a human penis and which is so shaped as to operationally encircle the penis by compression, the penis internally including a urethral duct in a lower position and two cavernous bodies in lateral positions, the device including a first part adapted to operationally exert an even compression force on the upper part of the penis, and a second part adapted to operationally exert direct compression forces on both cavernous bodies and indirect compression forces on the urethral duct, induced by the direct compression forces exerted on both cavernous bodies, so as to achieve a stable lateral compression for closing the urethral duct.

### FIELD OF THE INVENTION

The present invention relates to a non-invasive device against urinary incontinence.

### BACKGROUND ART

So far, no effective, safe, non-invasive and non-traumatic remedy has been found to the problem of male incontinence.

In fact, adult diapers or sanitary napkins are still the items most commonly used to counter the effects of incontinence. However, it is clear that the use of absorbing materials and/or devices cannot be considered to be a solution to the problem.

Narrowing the research to systems aimed at solving the problem of male incontinence in a non-invasive, totally non-traumatic manner, diapers excluded, numerous devices are known in the art which simply compress the user's penis by exerting additional pressure on the urethral duct. Such pressure, which is aimed at closing the urinary duct, can be obtained by using a special geometry directly pressing against the urethral duct or via a mechanical action exerted by movable or semi-movable parts that can be actuated by screwing or compression.

This direct pressure against the urethral duct necessarily creates a point of unstable equilibrium for the penis inside the device. When the user makes movements that are common in everyday life, such as picking up keys from the ground, getting out of a car or standing up from a chair, such instability translates into a shift of the penis to the right or to the left of the initial point of equilibrium of the device, thereby causing the urinary duct to open and urine to leak out.

If this were not true, there would be no reason to use diapers.

The invasive anti-incontinence devices already available will not be taken into account herein, since they are difficult to use and subject the urinary duct to a strong risk of infection (typically by Escherichia Coli).

Due to the instability shown by the non-invasive devices against male incontinence currently known in the art, such devices cannot be considered to be functional and effective.

### SUMMARY OF THE INVENTION

It is therefore one object of the present invention to provide a non-invasive device against urinary incontinence which can overcome all of the above-mentioned problems.

Following studies and practical tests, a device has been defined which, thanks to its own particular geometry, considerably improves the reliability of this type of non-invasive anti-incontinence devices.

The device according to the invention has a geometry that, unlike prior-art solutions, does not exert pressure directly on the urethral duct, but on the cavernous bodies that surround the urethral duct.

This pressure exerted on the cavernous bodies is indirectly transferred to the walls of the urethral duct, which will thus tend to close the urinary duct.

The advantage of the present invention lies in the fact that it ensures stability of the penis in the correct position also during and/or following everyday movements and the like.

Compared to prior-art devices, the innovation brought about by this invention consists of transforming a point of unstable equilibrium (as is typical of prior-art devices) into a point of stable equilibrium, delegating the task of pressing against the urethral duct to the cavernous bodies, thereby preventing urine from leaking out. The urethral duct is thus compressed indirectly via direct pressure exerted on the cavernous bodies.

The present invention relates to a non-invasive device against urinary incontinence, adapted to be applied to the external part of a human penis and so shaped as to operationally encircle the penis by compression, said penis internally comprising a urethral duct in a lower position and two cavernous bodies in lateral positions, the device being characterized in that it comprises a first part adapted to operationally exert an even compression force on the upper part of the penis, and a second part adapted to operationally exert direct compression forces on both cavernous bodies and indirect compression forces on the urethral duct, induced by said direct compression forces exerted on both cavernous bodies, so as to achieve a stable lateral compression for closing the urethral duct.

In particular, the present invention relates to a non-invasive device against urinary incontinence as specifically set out in the claims, which are an integral part of the present description.

### BRIEF DESCRIPTION OF THE DRAWINGS

Further objects and advantages of the present invention will become apparent from the following detailed description of a preferred embodiment (and variants thereof) referring to the annexed drawings, which are only supplied by way of non-limiting example, wherein:

FIG. 1 shows a side view of one embodiment of the device according to the invention;

FIG. 2 shows one embodiment of the inner part of the device;

FIGS. 3 and 4 show side views of the device of FIG. 1;

FIG. 5 is a cross-sectional view of a penis;

FIG. 6 is an explanatory graph showing some examples of the curvilinear profile of the lower inner part of the device;

FIG. 7 schematically shows how the lines of force developed by the device can compress the penis.

In the drawings, the same reference numerals and letters identify the same items or components.

### DETAILED DESCRIPTION OF SOME EMBODIMENTS OF THE INVENTION

Once worn, the device exerts an even pressure on the upper part of the penis; this pressure is then transferred to the lower part of the penis, pressing against the geometry of the lower part of the device.

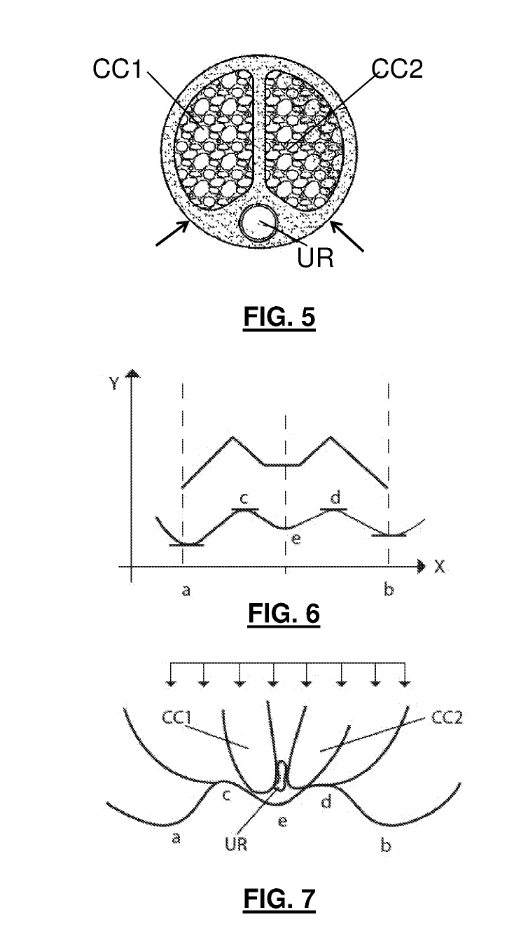

The above-mentioned upper and lower parts of the penis and of the device refer to the shape of the penis cross-section as shown in FIG. 5, which highlights the urethral duct UR at the bottom and two cavernous bodies CC1, CC2 at the sides.

The device 1 is provided in the form of a band encircling the penis in a firm and removable manner, which comprises at least one reclosable opening allowing the device to be arranged in such a way that the penis will remain operationally compressed by it.

The device comprises an upper part 2 and a lower part 3 opposite to each other. Furthermore, in one embodiment the lower and upper parts are fixedly connected together on one side 4, whereas on the other side they are connected by closing means, e.g. two flaps 5′, 5″ adapted to be overlapped for closing and opening the device around the penis. Closing can be effected by means of Velcro strips or other systems such as soft plastic ties for fishbone or saw-tooth or rack-type fastening.

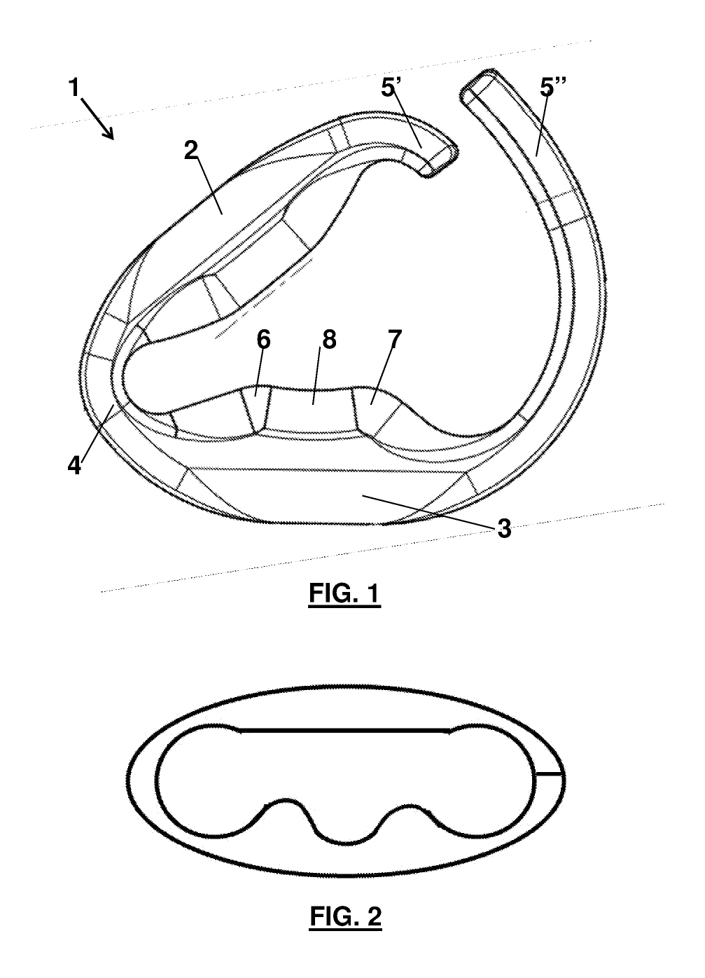

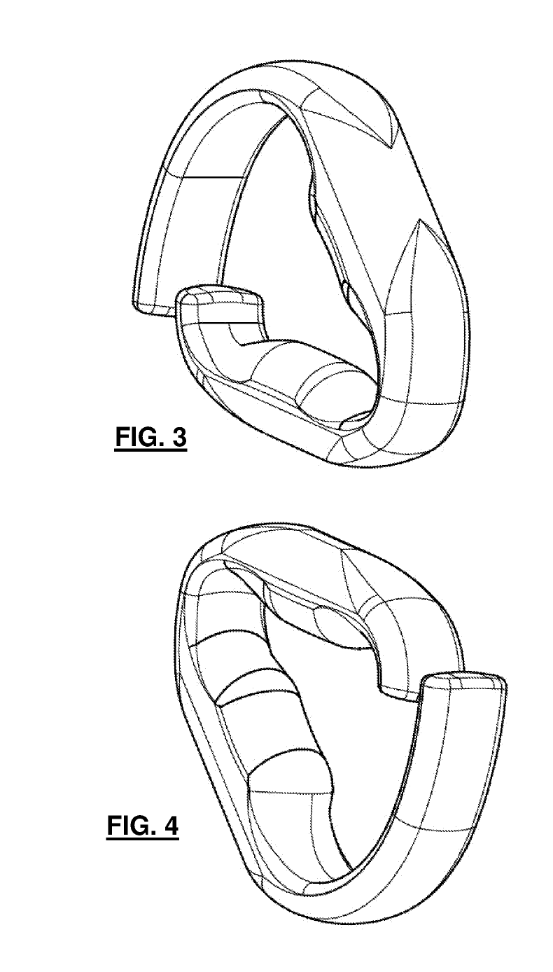

The device preferably comprises a rigid inner layer, e.g. shaped as shown in FIG. 2, coated with a soft foam material part, preferably of antibacterial nature, to adapt itself to the shape of the penis, thus having an overall shape like the one shown by way of example in FIGS. 1, 3, 4.

One important aspect of the invention is the geometry of the profile of the lower part 3 of the device. It has a curvilinear shape. As shown in FIGS. 6 and 7, an area is defined between two lateral points of absolute minimum a and b, between which two points of relative maximum c and d are defined, with a point of relative minimum e in between. This can be achieved by means of curvatures or broken lines of the rigid inner material, the structure being then effectively modelled by the soft external material.

The exemplary curves drawn on the x-y axes, where a<x<b, highlight a curvature having points of horizontal tangency (derivative=0), or a broken line with inward points of maximum and minimum. As aforesaid, a and b are points of absolute minimum, e is a point or area of relative minimum in a substantially central position relative to c and d, which are points or areas of relative maximum.

Point e is a point of stable equilibrium because it is contoured by c and d points, as opposed to prior-art solutions, wherein the central point is a point of unstable equilibrium directly compressing the urethral duct.

The two lateral protuberances 6, 7 exert a pressure with a lateral force component towards the cavernous bodies (positioned at the sides of the urethral duct), which, being affected by such field of forces orthogonally to the curve surface, close and compress the urethral duct in between. It can be noticed (FIG. 7) that the urethral duct UR is compressed by a vector field of forces that is different from the one achievable with prior-art devices: the upper part of the device exerts an even field of forces on the penis, while the lower part, instead of compressing the urethral duct UR directly from below, compresses it laterally and indirectly, i.e. compression is exerted directly and laterally on the cavernous bodies CC1, CC2 by lateral force components, causing the urethral duct to become deformed between the two cavernous bodies.

Such field of forces (or pressures) generated by the specific geometry of this device ensures that the urethral duct will stay properly closed even during movements, since the penis will be at a point of stable equilibrium, unlike current anti-incontinence devices, wherein the initial position is a point of unstable equilibrium.

Therefore, the basic idea is to exert lateral pressure on the cavernous bodies beside the urethral duct, thus creating a point of stable equilibrium to exert pressure on the urethral duct. This provides stability, comfort of use and uninterrupted efficiency.

The rigid inner body (FIG. 2) is made, for example, of thermoplastic material, preferably Nylon 6 (PA 6).

As alternatives to Nylon 6, the following materials may be employed: PP, PE, PA11, PA12, PA66, PA6-66, PC, ABS, PEEK, rigid PVC, plasticized PVC, TPU, or any thermoplastic polymeric material having a sufficient modulus of elasticity (>40 MPa of tensile modulus) for creating the inner structure, or blends thereof.

Said rigid component represents the inner part of the device.

Said component is manufactured by injection moulding.

Externally to said rigid component, a layer of soft material is applied by overmoulding or coating; the layer of soft material is preferably made of antibacterial thermoplastic foam material.

Such soft, elastic and light coating can also be obtained by dipping, spraying, binding, deposition or the like, and its thickness is in the range of 0.5 to 10 mm depending on the mechanical characteristics of the material used for said coating.

Other materials may also be used for such coating: EVA polymer foam, SEBS foam, SBS foam, TPU foam, PS foam, polyisoprene, latex, silicone and other thermoplastic or thermosetting polymers, or blends thereof.

Open-cell or closed-cell foam materials are to be preferred for this purpose, due to their lower weight as well as their lower environmental impact (and disposal costs).

As previously mentioned, the device includes a fastening system that can be modulated and adapted to each user; for example, closure adjustment can be provided by Velcro strips, but other adjustable closing systems can be adopted as well, whether integral with or separate from the coating geometry.

The above-described non-limiting example of embodiment may be subject to variations without departing from the protection scope of the present invention, including all equivalent designs known to a man skilled in the art.

The elements and features shown in the various preferred embodiments may be combined together without however departing from the protection scope of the present invention.

The advantages deriving from the application of the present invention are apparent.

This device has been developed, tested and validated by means of practical tests to ensure the absence of leaks in individuals suffering from male incontinence. Clear benefits to the user's life style and quality are easily imaginable, since the use of diapers is avoided. The numerous advantages also include a smaller environmental impact of this device as concerns the costs and the pollution related to disposal and production of traditional disposable diapers.

In fact, due to its innovative antibacterial component and washable materials, this device can be offered as a reusable, as opposed to disposable, device.

It is important to underline that this solves the problem of the point of unstable equilibrium, which is characteristic of prior-art solutions, and which gives rise to problems, e.g. caused by the person's movements, that can cause the pressure element to shift outside the area of the urethral duct, thus making the device ineffective or even harmful, since it can only be effective in particular conditions, e.g. when the person is still, e.g. sitting in a wheel chair or lying in bed, such effectiveness disappearing if the person moves.

From the above description, those skilled in the art will be able to produce the object of the invention without introducing any further construction details.

1. A non-invasive device against urinary incontinence, adapted to be applied to the external part of a human penis and so shaped as to operationally encircle the penis by compression, said penis internally comprising a urethral duct in a lower position and two cavernous bodies in lateral positions, the device being characterized in that it comprises a first part adapted to operationally exert an even compression force on the upper part of the penis, and a second part adapted to operationally exert direct compression forces on both cavernous bodies and indirect compression forces on the urethral duct, induced by said direct compression forces exerted on both cavernous bodies, so as to achieve a stable lateral compression for closing the urethral duct.

2. The device according to claim 1, wherein said second part comprises a curvilinear inner profile, with two points or areas of relative maximum, with a point or area of relative minimum in between, said points or areas of relative maximum being adapted to exert said direct compression forces on the two cavernous bodies, respectively, said direct compression forces comprising a lateral component.

3. The device according to claim 1, comprising an inner part made of rigid material and an outer part made of soft material, which covers said inner part.

4. The device according to claim 3, wherein said rigid inner part is made of thermoplastic material, or PP, PE, PA11, PA12, PA66, PA6-66, PC, ABS, PEEK, rigid PVC, plasticized PVC, TPU, or blends thereof.

5. The device according to claim 3, wherein said outer part is made by dipping, or spraying, or binding, or deposition, and comprises materials such as EVA polymer foam, SEBS foam, SBS foam, TPU foam, PS foam, polyisoprene, latex, silicone and other thermoplastic or thermosetting polymers, or blends thereof.

6. The device according to claim 1, comprising at least one reclosable opening adapted to allow positioning the device in a manner such that the penis will remain operationally compressed.

7. The device according to claim 6, wherein said at least one reclosable opening comprises closing means adapted to be operationally overlapped for closing and opening the device around the penis.

8. The device according to claim 6, wherein said at least one reclosable opening is implemented by means of Velcro strips, or soft plastic ties for fishbone or saw-tooth or rack-type fastening.

9. The device according to claim 3, wherein, in said inner part, said second part has a curvilinear inner profile comprising bends or broken lines.
